(19) United States
(12) Patent Application Publication
(10) Pub. No.: US 2021/0315552 A1
(43) Pub. Date: Oct. 14, 2021

(54) BIOLOGICAL SAMPLE COLLECTION INSTRUMENTS

(71) Applicant: Carbon, Inc., Redwood City, CA (US)

(72) Inventors: Hardik Kabaria, Redwood City (US); Owen Lu, San Mateo, CA (US); Joseph M. DeSimone, Monte Sereno, CA (US); Shawn Fortner, Redwood City, CA (US)

(21) Appl. No.: 17/220,083

(22) Filed: Apr. 1, 2021

Related U.S. Application Data
(60) Provisional application No. 63/009,777, filed on Apr. 14, 2020.
(60) Provisional application No. 63/016,647, filed on Apr. 28, 2020.

(51) Int. Cl. A61B 10/02; B33Y 10/00; B33Y 70/00; B33Y 80/00

(52) U.S. Cl. CPC A61B 10/02; B33Y 10/00; A61B 2010/0225; B33Y 80/00; A61B 2560/0406; B33Y 70/00

(57) ABSTRACT

A biological specimen collection instrument configured for collecting a biological specimen to be analyzed includes: (a) a handle; (b) an elongate flexible or elastomeric stem extending from said handle, said stem having a distal portion terminating at a tip; and (c) a flexible or elastomeric lattice collection element connected to said stem distal portion, said lattice collection element having a body portion and a distal end portion, with at least said body portion, and optionally said distal end portion, having openings therein in a configuration that forms at least one biological specimen collection space.

### FIELD OF THE INVENTION

The present invention concerns biological sample collection instruments, sometimes known as “swabs,” and methods of making the same.

### BACKGROUND OF THE INVENTION

An effective biological sample collection instrument must meet a variety of requirements. The instrument should be sufficiently strong to make good physical contact with a surface from which a sample is being collected. Yet, when used to collect a sample from a patient, it should also be sufficiently flexible to be comfortable, and sufficiently flexible to reach a surface, such as a nasopharyngeal mucosal surface, for which there is not easy linear access. And not only must the instrument pick up a sufficient quantity of biological material, but it must effectively release the collected material into laboratory reagents for analysis.

For example, nasopharyngeal swabs have been known for at least a quarter century for testing for viral infection. See, e.g., A. Liav et al., U.S. Pat. No. 5,252,458; J. Wiselka et al., Epidemiol. Infect. 111, 337-346 (1993). However, when traditional fiber swabs are used for such procedures, the biological sample can be absorbed into the fiber, making its release into reagents for analytical testing more difficult. Flocked swabs (that is, swabs produced by electrostatically depositing fibers onto an adhesive coated tip; see, e.g., U.S. Pat. Nos. 8,114,027; 8,317,728; 8,979,784; 9,011,358; and 9,173,779) have been shown to provide better release of certain biological samples, but the supply of such swabs can be limited when demand is high, possibly due to supply chain problems and/or the added complexity of the electrostatic flocking process. Accordingly, there is a need for new approaches to making collection instruments such as nasopharyngeal swabs.

### SUMMARY OF THE INVENTION

It is known that small brushes can be produced by additive manufacturing (see, e.g., U.S. Pat. No. 8,172,473), and when a shortage of nasopharyngeal swabs became apparent during the COVID-19 pandemic, a number of different designs for additively manufactured nasopharyngeal swabs were proposed, including the bristle design, the honeydipper design, the cattail design, and the brush design (see generally R. Arnout, Covid 19 Swab Summary (GitHub 25 Mar. 2020) and J. Ford, Covid 19 USF Swab Summary (GitHub 27 Mar. 2020)). We propose a lattice design for an additively manufactured swab as an instrument that provides a good balance of sample collection volume, stiffness, flexibility, and sample releasability.

In some embodiments, a biological specimen collection instrument is configured for collecting a biological specimen to be analyzed. The instrument includes (a) a handle; (b) an elongate flexible or elastomeric stem extending from the handle, the stem having a distal portion terminating at a tip; and (c) a flexible or elastomeric lattice collection element connected to the stem distal portion. The lattice collection element has a body portion and a distal end portion, with at least the body portion, and optionally the distal end portion, having openings therein in a configuration that forms at least one biological specimen collection space.

In some embodiments, the lattice collection element is cylindrical, spherical, or oblong in shape, and/or has at least one generally planar collection surface formed thereon, and/or has annular or longitudinal grooves or ridges on a surface thereof.

In some embodiments, the lattice collection element is connected directly to the stem distal element, and the instrument further includes (d) a plurality of flexible or elastomeric branches connected to the stem distal portion and radiating outward therefrom, with the lattice collection element connected to the branches.

In some embodiments, the stem distal portion extends at least partially into the lattice collection element body portion to form a stiffening core therein.

In some embodiments, the stem distal portion in the lattice collection element body portion tapers progressively to the tip thereof.

In some embodiments, the stem distal portion in the lattice collection element body portion is helically shaped.

In some embodiments, the stem distal portion is positioned in, and optionally but in some embodiments preferably connected to, the lattice collection element distal end portion.

In some embodiments, the lattice collection element is comprised of a plurality of interconnected struts, optionally but in some embodiments preferably with the struts configured in a pattern of repeating unit cells (e.g., hexagonal unit cells), and optionally a density of the interconnected struts that decreases at the distal end portion.

In some embodiments, the lattice collection element is comprised of a triply periodic surface lattice (e.g., a gyroid lattice).

In some embodiments, the lattice collection element comprises a conformal lattice.

In some embodiments, the lattice collection element tapers progressively along the body portion to the distal end portion.

In some embodiments, the lattice collection element distal end portion is domed.

In some embodiments, the lattice collection element is from 1 or 2 millimeters in diameter, to 4 or 5 millimeters in diameter (preferably 3 millimeters in diameter) and is from 1 centimeter in length, to 2 or 3 centimeters in length (preferably 1.5 centimeters in length).

In some embodiments, the instrument is configured as a nasopharyngeal swab, a mid-turbinate swab, or an anterior nares swab.

In some embodiments, at least the lattice collection element, the branches when present, the stiffening core when present, optionally the stem, and optionally the handle, are produced together by photopolymerization of a resin in an additive manufacturing process.

In some embodiments, the instrument includes a stop connected to the handle (e.g., for a mid-turbinate swab), the stop optionally but in some embodiments preferably having openings extending therethrough to facilitate flow of resin therethrough during additive manufacturing of the instrument.

According to some embodiments, an assembly includes a sacrificial connector and a plurality of instruments as described herein connected by the handle of each thereof to the sacrificial connector. The sacrificial connector optionally, but in some embodiments preferably, has a unique identifier thereon.

According to some embodiments, an instrument or assembly as described herein comprises a polymer having: a Young's modulus of from 10 or 20 megapascals, to 1,000 or 2,000 megapascals, at a temperature of 25 degrees Centigrade; and/or an elongation at break of from 50, 100, or 200 percent, up to 300, 500, or 1,000 percent, at a temperature of 25 degrees Centigrade.

According to some embodiments, a method of making an instrument or assembly as described herein includes (a) providing a light-polymerizable resin; and (b) producing the instrument, or at least the lattice collection element thereof (including the branches and the core when present) or assembly from the resin by an additive manufacturing process.

In some embodiments, the additive manufacturing process comprises top-down or bottom-up stereolithography (e.g., continuous liquid interface production).

In some embodiments, the instrument or assembly is formed on a build platform, with the handle or the sacrificial connector adhered to the build platform, and the shaft extending perpendicularly away therefrom.

In some embodiments, a plurality of the instrument or the assembly is formed simultaneously on the build platform during the producing step.

In some embodiments, the method further includes (c) washing the instrument or assembly; (d) optionally but in some embodiments preferably further curing the instrument or assembly; (e) optionally but in some embodiment preferably sterilizing the instrument or assembly; and then (f) optionally but in some embodiments preferably aseptically packaging the instrument.

While the present invention has been described primarily with reference to nasopharyngeal swabs, it will be appreciated that the instruments described herein can be adapted to other purposes, such as oropharnyngeal swabs buccal swabs, cervical swabs, forensic testing swabs such as for crime scene analysis, etc.

The foregoing and other objects and aspects of the present invention are explained in greater detail in the drawings herein and the specification set forth below. The disclosures of all United States patent references cited herein are to be incorporated herein by reference.

### BRIEF DESCRIPTION OF THE DRAWINGS

FIG. 1A is a side view of a cylindrical lattice portion of an instrument as described herein.

FIG. 1B is a partially cut-away view of the cylindrical lattice of FIG. 1A.

FIG. 2A is a side view of a second embodiment of a cylindrical lattice portion of an instrument as described herein.

FIG. 2B is a partially cut-away view of the cylindrical lattice of FIG. 2A.

FIG. 3A is a side view of a third embodiment of a cylindrical lattice portion of an instrument as described herein.

FIG. 3B is a partially cut-away view of the cylindrical lattice of FIG. 3A.

FIG. 4 illustrates an assembly comprising a plurality of instruments as described herein joined to a sacrificial connector.

FIG. 5 illustrates a variety of different stem distal portions, or stiffening cores, as may be used in an instrument as described herein.

FIG. 6 is a detailed perspective view of a cylindrical lattice portion of an instrument as described herein.

FIGS. 7A-7D are views of an instrument as described herein embodied as a mid-turbinate swab.

FIG. 8 is a detailed view of an alternate embodiment of a lattice collection element for a collection instrument such as a mid-turbinate swab.

FIG. 9 schematically illustrates one embodiment of how a lattice collection element produced by additive manufacturing may be joined to a separately produced handle and stem.

### DETAILED DESCRIPTION OF ILLUSTRATIVE EMBODIMENTS

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The same numbers are assigned to corresponding or analogous elements in different embodiments shown in the Figures, for the sake of simplicity.

As used herein, the term “and/or” includes any and all possible combinations of one or more of the associated listed items, as well as the lack of combinations when interpreted in the alternative (“or”).

1. Additive Manufacturing.

Techniques for producing objects from light-polymerizable resins are known and include bottom-up and top-down additive manufacturing, generally known as stereolithography. Such methods are known and described in, for example, U.S. Pat. No. 5,236,637 to Hull, U.S. Pat. Nos. 5,391,072 and 5,529,473 to Lawton, U.S. Pat. No. 7,438,846 to John, U.S. Pat. No. 7,892,474 to Shkolnik, U.S. Pat. No. 8,110,135 to El-Siblani, U.S. Patent Application Publication No. 2013/0292862 to Joyce, and US Patent Application Publication No. 2013/0295212 to Chen et al. The disclosures of these patents and applications are incorporated by reference herein in their entirety.

In some embodiments, the additive manufacturing step is carried out by one of the family of methods sometimes referred to as continuous liquid interface production (CLIP). CLIP is known and described in, for example, U.S. Pat. Nos. 9,211,678; 9,205,601; 9,216,546; and others; in J. Tumbleston et al., Continuous liquid interface production of 3D Objects, Science 347, 1349-1352 (2015); and in R. Janusziewcz et al., Layerless fabrication with continuous liquid interface production, Proc. Natl. Acad. Sci. USA 113, 11703-11708 (Oct. 18, 2016). Other examples of methods and apparatus for carrying out particular embodiments of CLIP include, but are not limited to: Batchelder et al., US Patent Application Pub. No. US 2017/0129169 (May 11, 2017); Sun and Lichkus, US Patent Application Pub. No. US 2016/0288376 (Oct. 6, 2016); Willis et al., US Patent Application Pub. No. US 2015/0360419 (Dec. 17, 2015); Lin et al., US Patent Application Pub. No. US 2015/0331402 (Nov. 19, 2015); D. Castanon, S Patent Application Pub. No. US 2017/0129167 (May 11, 2017). B. Feller, US Pat App. Pub. No. US 2018/0243976 (published Aug. 30, 2018); M. Panzer and J. Tumbleston, US Pat App Pub. No. US 2018/0126630 (published May 10, 2018); K. Willis and B. Adzima, US Pat App Pub. No. US 2018/0290374 (Oct. 11, 2018) L Robeson et al., PCT Patent Pub. No. WO 2015/164234 (see also U.S. Pat. Nos. 10,259,171 and 10,434,706); and C. Mirkin et al., PCT Patent Pub. No. WO 2017/210298 (see also US Pat. App. US 2019/0160733).

Resins, Any suitable resin can be used for carrying out the present invention, including but not limited to those described J. Rolland, K. Chen, J. Poelma, J. Goodrich, R. Pinschmidt, J. DeSimone, and L. Robeson, Methods of producing three-dimensional objects from materials having multiple mechanisms of hardening U.S. Pat. No. 9,676,963 (Jun. 13, 2017), and in U.S. Pat. Nos. 10,239,255; 10,316,213; and others. The resin may be chosen to create an instrument comprised of a polymer having tensile properties preferred for the particular end use of the instrument. For example, in some embodiments, the resin may be selected so that the instrument or assembly is comprised of a polymer having a Young's modulus of from 10 or 20 megapascals, to 1,000 or 2,000 megapascals, at a temperature of 25 degrees Centigrade; and/or an elongation at break of from 50, 100, or 200 percent, up to 300, 500, or 1,000 percent, at a temperature of 25 degrees Centigrade.

2. Collection Instruments and Assemblies.

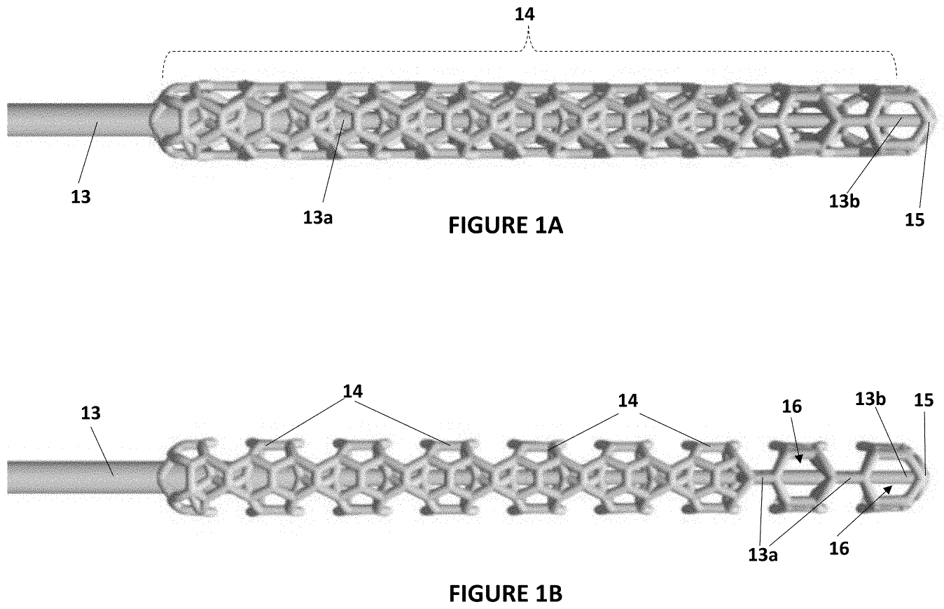

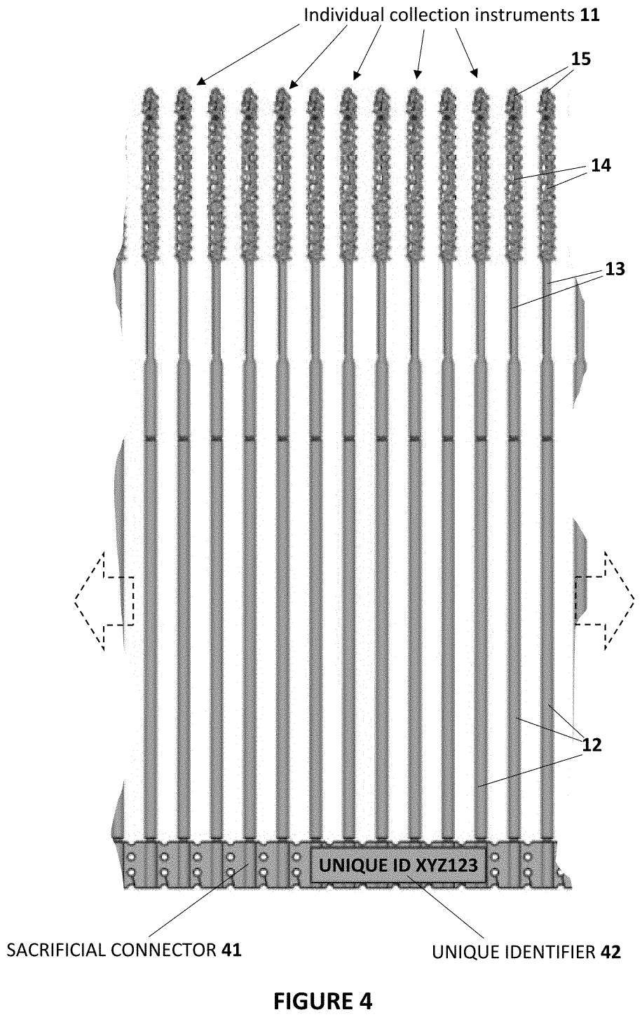

A first embodiment of a biological specimen collection instrument configured for collecting a biological specimen to be analyzed is given in FIGS. 1A-1B. The instrument includes a handle (12) (shown in FIG. 4); an elongate flexible or elastomeric stem (13) extending from the handle, the stem having a distal portion (13a) terminating at a tip (13b); and a flexible or elastomeric lattice collection element connected to the stem distal portion, the lattice collection element having a body portion (14) and a distal end portion (15), with at least the body portion, and optionally the distal end portion, having openings (16) therein in a configuration that forms at least one biological specimen collection space. While in the illustrative embodiment the lattice collection element is cylindrical, it may take any suitable shape depending on the specific purpose for which the instrument is intended. Including spherical or oblong in shape. One or more generally planar collection surfaces (not shown) can be formed thereon, and one or more annular or longitudinal grooves or ridges (also not shown) can be included on a surface thereof.

As best seen in FIG. 6, the instrument may include a plurality of flexible or elastomeric branches (17) connected to the stem distal portion and radiating outward therefrom, with the lattice collection element connected to the branches. The branches may take any suitable configuration, including substantially linearly as shown, or curve or taper to form ribs running substantially parallel to surface of lattice, on surface or within the lattice, or any other suitable configuration.

In the illustrative embodiment of FIGS. 1A-1B and FIG. 6, the stem distal portion extends at least partially into the lattice collection element body portion to form a stiffening core therein. While in some embodiments this stiffening core is aligned with the center axis of the lattice body portion, in other embodiments it could be offset from the center axis, and/or the stem could split into two or more branches that interconnect with the lattice.

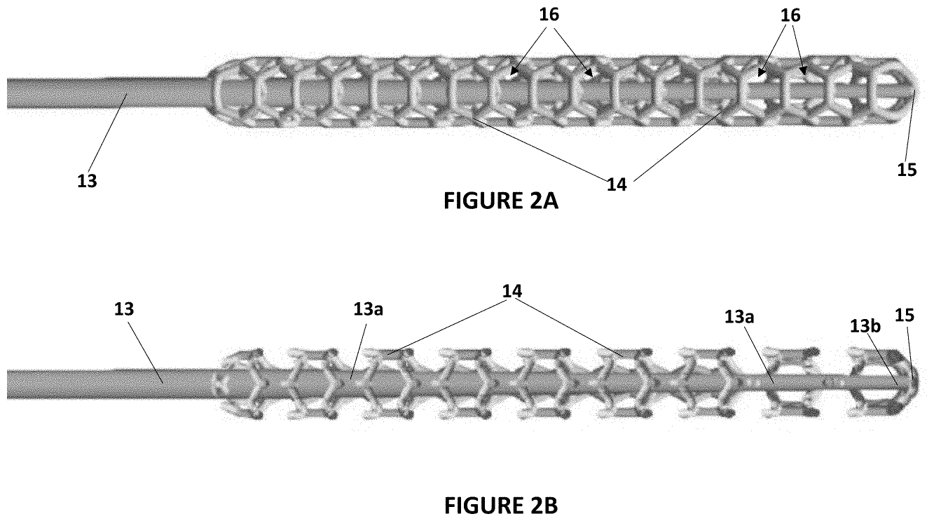

The instrument of FIG. 2A-2B is similar to that of FIG. 1A-1B, except that now the stem distal portion in the lattice collection element body portion tapers progressively to the tip thereof (e.g., to reduce surface creasing or “kinking” of the lattice when it is turned in a curve, as may occur during collection of a sample from a nasopharyngeal surface).

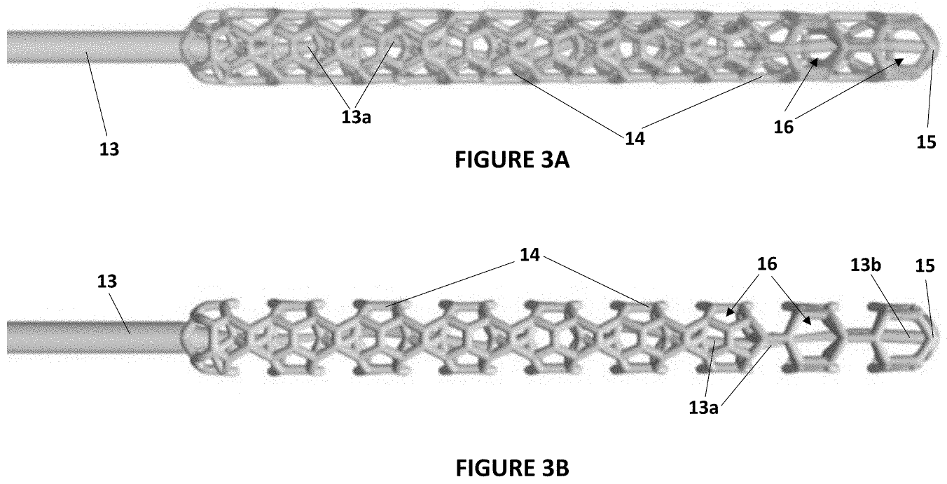

The instrument of FIG. 3A-3B is likewise similar to that of FIG. 1A-1B, except that now the stem distal portion in the lattice collection element body portion is helically shaped (again, to reduce surface creasing or “kinking” of the lattice when it is turned in a curve, as may occur during collection of a sample from a nasopharyngeal surface).

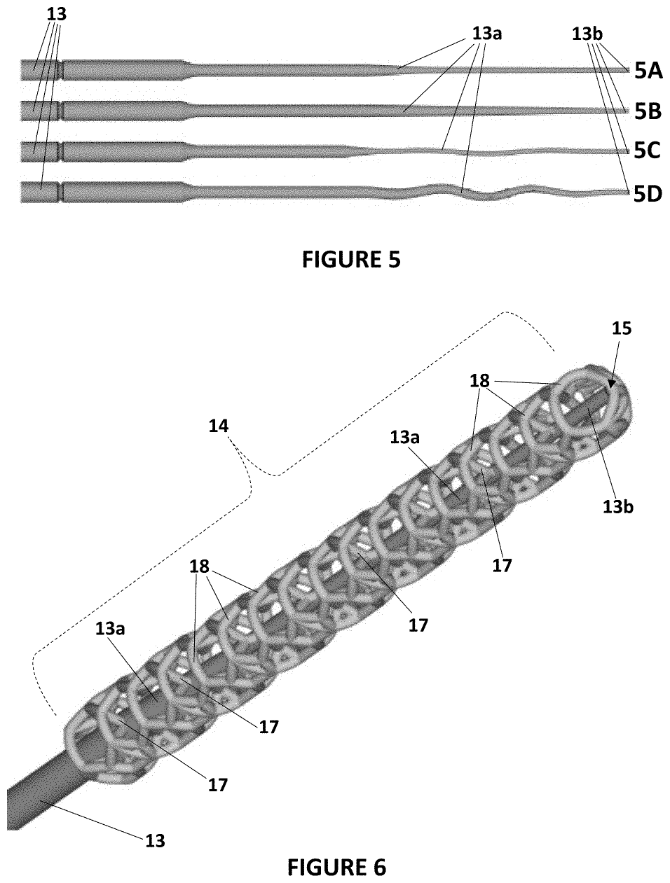

For greater clarity, FIGS. 5A-5D show various shapes for stiffening cores free of any branches or lattice collection elements.

In the above non-limiting embodiments, it is seen that the stem distal portion is positioned in, and optionally but in some embodiments preferably connected to, the lattice collection element distal end portion.

In some of the above non-limiting embodiments, the lattice collection element tapers progressively along the body portion to the distal end portion, and/the lattice collection element distal end portion is domed.

While instruments described herein may be configured for a variety of purposes as noted above, in some embodiments they are configured as a nasopharyngeal swab, a mid-turbinate swab, or an anterior nares swab. The swab may be pre-dimensioned as appropriate for the particular application, and/or for use in infant, juvenile, or adult human patients. For patients with non-standard anatomical features the swab may be custom dimensioned and manufactured. In some embodiments, the lattice collection element is from 1 or 2 millimeters in diameter, to 4 or 5 millimeters in diameter (preferably 3 millimeters in diameter) and is from 1 centimeter in length, to 2 or 3 centimeters in length (preferably 1.5 centimeters in length).

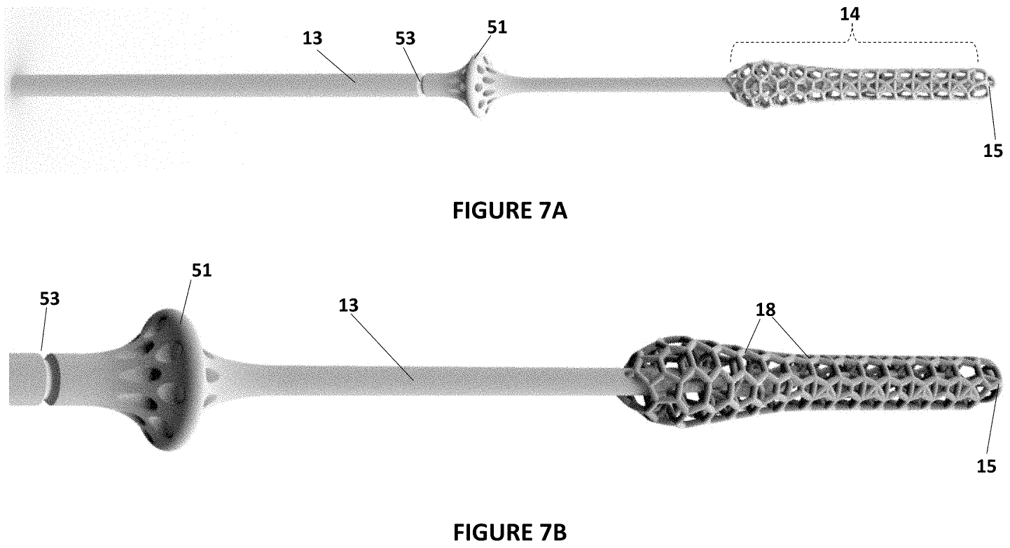

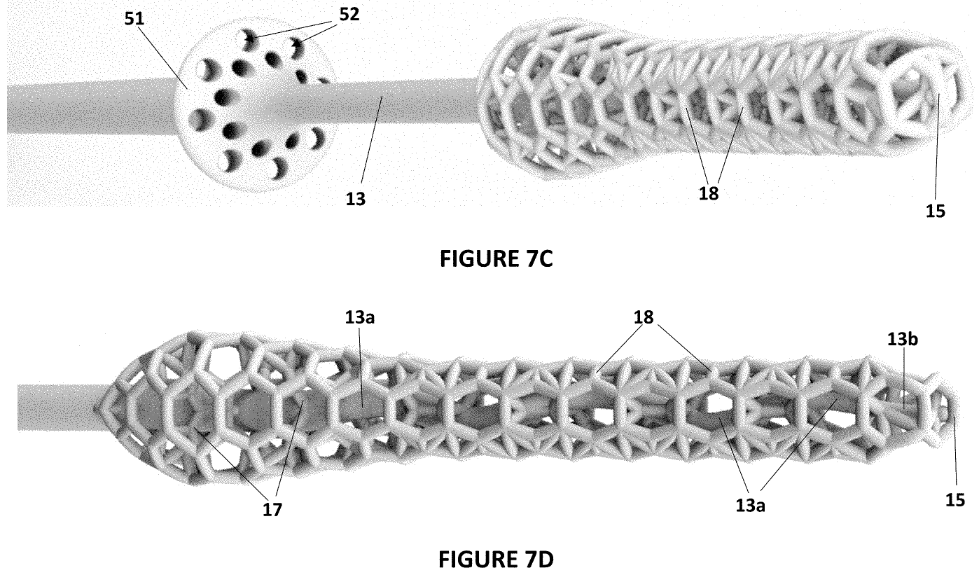

A first example of a mid-turbinate swab is given in FIGS. 7A-7D. In addition to the elements and features described in connection with a nasopharyngeal swab above, the mid-turbinate swab may include a stop (51) on the handle thereof, configured to mark a desired insertion depth into the nasal passages of the subject. The stop may have one or more orifices or openings (52) therein, these included to facilitate the flow of resin through the stop in a manner that speeds production by additive manufacturing. Finally, the stem or handle may include a fracturable score or break point (53), above or below the stop if present, by which the handle, and optionally a portion of the stem, can be separated from the collection element when the collection element is deposited into a container for further transport or analysis.

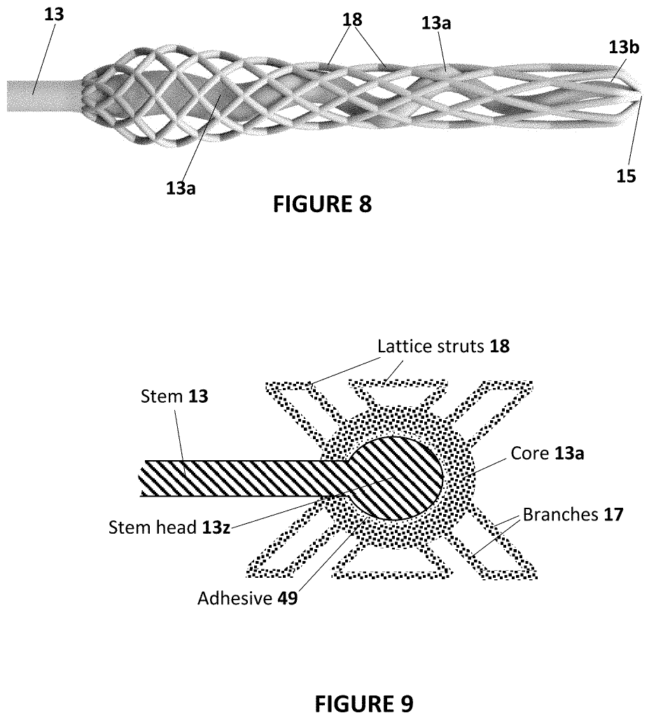

An alternate embodiment of a lattice collection element for a mid-turbinate swab is shown in FIG. 8. In this embodiment, which employs a tapered, helical, core 13a such as described above, note that the struts 18 of the lattice collection element is connected directly to the core, rather than through interconnecting branches.

In the illustrated embodiments, the lattice collection element is comprised of a plurality of interconnected struts (18), optionally but in some embodiments preferably with the struts configured in a pattern of repeating unit cells (e.g., hexagonal unit cells). In some embodiments, the density of the interconnected struts decreases at the distal end portion (that is, the lattice becomes more “open.”). In other embodiments (not shown) the lattice collection element is comprised of a triply periodic surface lattice (e.g., a gyroid lattice). In some embodiments, the lattice collection element may comprise a conformal lattice.

As shown in FIG. 4, the instruments described herein can conveniently be supplied as an assembly comprising a sacrificial connector (41) and a plurality of instruments (11) connected by the handle of each thereof to the sacrificial connector, the sacrificial connector optionally, but in some embodiments preferably, having a unique identifier (42) thereon. The unique identifier can be any suitable identifier, including an identification number, code, bar code, or QR code and may include or be associated with information about the assembly and/or the instruments, such as a date of manufacture, a type of instrument, instrument parameters and the like.

3. Methods of Making.

Instruments or assemblies as described above may be produced by (a) providing a light-polymerizable resin; and (b) producing the instrument or assembly from the resin by an additive manufacturing process (e.g., top-down or bottom-up stereolithography such as described above. Where the instrument or assembly is produced on a build platform, the handle or the sacrificial connector is preferably adhered to the build platform, with the shaft extending perpendicularly away therefrom, to enhance the density at which multiple copies may be produced simultaneously on the same platform. Thus it is preferred that, a plurality of the instrument or the assembly are formed simultaneously on the build platform during the producing step.

Depending on the resin chosen, after their additive manufacture the instruments or assembly can be washed and further cured (e.g., by baking) in accordance with known procedures such as described in W. McCall, J. Rolland, and C. Converse, Wash liquids for use in additive manufacturing with dual cure resins U.S. Pat. No. 10,343,331, (Jul. 9, 2019).

In some embodiments, the entire instrument is made by additive manufacturing. In other embodiments, only the lattice collection element (along with branches and core therein, when present) is made by additive manufacturing, and it can be joined to a pre-formed handle and stem by interference fit into a terminal portion of the stem, with an adhesive, by baking or further curing of the additively manufactured collection element onto the stem, or a combination thereof. (an example of which is given in FIG. 9, where the core 13a includes a cavity into which an enlarged terminal stem head 13z may be inserted, through an opening smaller than the enlarged head to provide an interference fit, optionally with adhesive 49). In still other embodiments the stem is formed by additive manufacturing along with the collection element, and the stem is joined (by any suitable technique) to a pre-formed, reusable or disposable, handle. Preformed handles, or preformed handles and stems, can be produced by any suitable technique, such as by injection molding.

The instruments may be sterilized before or after separating from the sacrificial connector (for example, by autoclaving, irradiating, contacting to ethylene oxide gas, etc.) and then packaged (preferably aseptically packaged) for use.

The foregoing is illustrative of the present invention, and is not to be construed as limiting thereof. The invention is defined by the following claims, with equivalents of the claims to be included therein.

We claim:

1. A biological specimen collection instrument configured for collecting a biological specimen to be analyzed, the instrument comprising:
(a) a handle;
(b) an elongate flexible or elastomeric stem extending from said handle, said stem having a distal portion terminating at a tip; and
(c) a flexible or elastomeric lattice collection element connected to said stem distal portion, said lattice collection element having a body portion and a distal end portion, with at least said body portion, and optionally said distal end portion, having openings therein in a configuration that forms at least one biological specimen collection space.

2. The instrument of claim 1, wherein said lattice collection element is cylindrical, spherical, or oblong in shape, and/or has at least one generally planar collection surface formed thereon, and/or has annular or longitudinal grooves or ridges on a surface thereof.

3. The instrument of claim 1, wherein:
said lattice collection element is connected directly to said stem distal element, or
said apparatus further comprises:
(d) a plurality of flexible or elastomeric branches connected to said stem distal portion and radiating outward therefrom, with said lattice collection element connected to said branches.

4. The instrument of claim 1, wherein said stem distal portion extends at least partially into said lattice collection element body portion to form a stiffening core therein.

5. The instrument of claim 4, wherein said stem distal portion in said lattice collection element body portion tapers progressively to said tip thereof.

6. The instrument of claim 4, wherein said stem distal portion in said lattice collection element body portion is helically shaped.

7. The instrument of claim 4, wherein said stem distal portion is positioned in, and optionally connected to, said lattice collection element distal end portion.

8. The instrument of claim 1, wherein said lattice collection element is comprised of a plurality of interconnected struts, optionally with said struts configured in a pattern of repeating unit cells, and optionally wherein a density of said interconnected struts decreases at said distal end portion.

9. The instrument of claim 1, wherein said lattice collection element is comprised of a triply periodic surface lattice.

10. The instrument of claim 1, wherein said lattice collection element comprises a conformal lattice.

11. The instrument of claim 1, wherein said lattice collection element tapers progressively along said body portion to said distal end portion.

12. The instrument of claim 1, wherein said lattice collection element distal end portion is domed.

13. The instrument of claim 1, wherein said lattice collection element is from 1 millimeter in diameter to 5 millimeters in diameter and is from 1 centimeter in length to 3 centimeters in length.

14. The instrument of claim 1, wherein said instrument is configured as a nasopharyngeal swab, a mid-turbinate swab, or an anterior nares swab.

15. The instrument of claim 1, wherein at least said lattice collection element, said branches when present, said stiffening core when present, optionally said stem, and optionally said handle, are produced together by photopolymerization of a resin in an additive manufacturing process.

16. The instrument of claim 1, further comprising a stop connected to said handle, the stop optionally having openings extending therethrough to facilitate flow of resin therethrough during additive manufacturing of said instrument.

17. An instrument of claim 1 comprised of a polymer having:
a Young's modulus of from 10 megapascals to 2,000 megapascals, at a temperature of 25 degrees Centigrade; and/or
an elongation at break of from 50 percent, up to 1,000 percent, at a temperature of 25 degrees Centigrade.

18. An assembly comprising:
a sacrificial connector; and
a plurality of biological specimen collection instruments configured for collecting a biological specimen to be analyzed, each of the instruments comprising:
(a) a handle;
(b) an elongate flexible or elastomeric stem extending from said handle, said stem having a distal portion terminating at a tip; and
(c) a flexible or elastomeric lattice collection element connected to said stem distal portion, said lattice collection element having a body portion and a distal end portion, with at least said body portion, and optionally said distal end portion, having openings therein in a configuration that forms at least one biological specimen collection space, the instruments connected by said handle of each thereof to said sacrificial connector, the sacrificial connector optionally having a unique identifier thereon.

19. A method of making a biological specimen collection instrument configured for collecting a biological specimen to be analyzed, the instrument comprising: (a) a handle; (b) an elongate flexible or elastomeric stem extending from said handle, said stem having a distal portion terminating at a tip; and (c) a flexible or elastomeric lattice collection element connected to said stem distal portion, said lattice collection element having a body portion and a distal end portion, with at least said body portion, and optionally said distal end portion, having openings therein in a configuration that forms at least one biological specimen collection space, the method comprising:
(a) providing a light-polymerizable resin;
(b) producing said instrument, or at least the lattice collection element thereof, including said branches and said core when present, or assembly from said resin by an additive manufacturing process.

20. The method of claim 19, wherein said additive manufacturing process comprises top-down or bottom-up stereolithography.

21. The method of claim 19, wherein said instrument or said assembly is formed on a build platform, with said handle or said sacrificial connector adhered to said build platform, and said shaft extending perpendicularly away therefrom.

22. The method of claim 21, wherein a plurality of said instrument or said assembly is formed simultaneously on said build platform during said producing step.

23. The method of claim 19, further comprising:
(c) washing said instrument or assembly;
(d) optionally further curing said instrument or assembly;
(e) optionally sterilizing said instrument or assembly; and then
(f) optionally aseptically packaging said instrument.
